(19) United States
(12) Patent Application Publication
(10) Pub. No.: US 2004/0071685 A1
(43) Pub. Date: Apr. 15, 2004

(54) Compositions and methods for increasing the bioavailability of plant polyphenols

(76) Inventors: Devin Houston, Siloam Springs, AR (US); Ken Paydon, Forsyth, MO (US); Steve Marr, Forsyth, MO (US); Rohit Medhekar, Springfield, MO (US)

Correspondence Address: Joseph A. Mahoney, Mayer, Brown, Rowe & Maw, P.O. Box 2828, Chicago, IL, 60090-2828, US

(21) Appl. No.: 10/268,304

(22) Filed: Oct. 9, 2002

(51) Int. Cl. A61K 38/47; A61K 9/64

(52) U.S. Cl. 424/94.61

(57) ABSTRACT

A composition and method for increasing the bioavailability of an aglycone in a subject. The composition comprises at least two enzymes, for example, a xylanase, a glucanase, or a glucosidase. A method for converting a glycosylated isoflavone into an aglycone in a digestive tract of a subject, comprising orally administering an effective amount of a composition comprising at least two enzymes, for example, a xylanase, a beta-glucanase, or glucosidase, and concomitantly administering a food stuff, for example, glycolsylated isoflavone.

### TECHNICAL FIELD

This invention relates to a composition and method for increasing the bioavailability of isoflavonoids and plant polyphenols in a subject.

### BACKGROUND

The general chemical term “glycoside” describes a carbohydrate (typically a sugar, more typically D-glucose) linked with a non-carbohydrate compound (termed “aglycone”). Glycosides are prevalent in nature and represent a significant portion of all the pharmacologically active constituents of botanicals.

In nature, this glycosidic linkage is thought to function as a sugar store, as well as to aid in transport of the biochemically active aglycone portion, because, as a class, the aglycones are much less water-soluble than their glycoside counterparts. The aglycone members of these compounds are attributed with critical biological plant functions, such as detoxification and defense.

The chemical structures of genistein, daidzein and glycitein are:

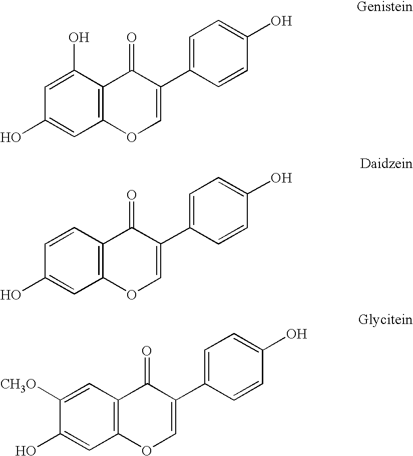

Substantial research revolves around the potential therapeutic benefits of these and other plant constituents. Isoflavones naturally occur in the glycoside form, more specifically as O-beta-D-glucosides. The enzyme believed to be responsible for the hydrolysis of these constituents into their respective glucose and aglycone portions is described as beta-D-glucosidase. This particular enzyme activity is endogenous to many of the fungally derived enzymes (sources including, but not limited to, A. niger, A. oryzae, A. awamori, Bacillus subtilis, Trichoderma longibrachiatum, etc.).

An example of the chemical reaction is given below:

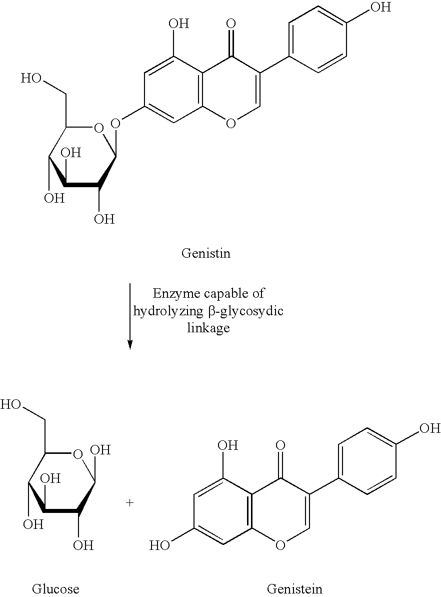

Isoflavones are absorbed in the gut; but the aglyconated forms are better absorbed than the natural glycosylated isoflavones found in foods. Therefore, there is a need to increase the enzymatic conversion of glycosylated isoflavones to the more readily bioavailable aglycone forms.

Many studies have shown that isoflavones, which are plant estrogens (phytoestrogens), play an important role in the prevention of cancer, cardiovascular disease and osteoporosis. Messina, M.; Persky, V.; Setchell, K. D. R.; Barnes, S. “Soy intake and cancer risk: A review of in vitro and in vivo data.” Nutr. Cancer, 21: 113-131 (1994); Andreson, J. W.; Johnstone, B. M.; Cook-Newell, M. E. “Meta-analysis of the effects of soy protein intake on serum lipids.” New Engl. J. Med., 333: 276-282 (1995); Kalu, D. N.; Masora, E. N.; Yu, B. P.; Hardin, R. R.; Hollis, B. W. “Modulation of age-related hyperparathyroidism and senile bone loss in Fischer rats by soy protein and food restriction.” Endocrinol., 122: 1847-1854 (1988); Blair, H. C.; Jordan, S. E.; Perterson, T. G.; Barnes, S. “Variable effects of tyrosine kinase inhibitors on avian osteoclastic activity and reduction of bone loss in ovariectomized rats.” J. Cell. Biochem., 61: issue 4, 629-637 (1996); Kao, P. C.; P'eng. F. K. “How to reduce the risk factors of osteoporosis in Asia?” Chinese Med. J., 55: 209-213 (1995); Messina, M. “Modern application for an ancient bean: soybeans and the prevention and treatment of chronic disease.” J. Nutr., 125: 567S-569S (1995). Most important and abundant among the plant estrogens found in soy are genistein and daidzein.

There are a number of hypotheses explaining the health benefits and the biochemical action of genistein and daidzein. Barnes et al. viewed genistein as a phytoestrogen that exhibits chemopreventive properties by acting as an estrogen antagonist. Barnes, S.; Grubbs, C.; Setchell, K. D. R.; Carlson, J. “Soybeans inhibit mammary tumors in models of breast cancer” in Mutagens and carcinogens in the diet, ed. Pariza, M.; Liss, A. R.; New York, pp. 239-253 (1990). The anti-cancer drug tamoxifen has a similar pharmacological effect. This basic hypothesis was further expanded by Akiyama et al., who showed that genistein is an excellent and specific inhibitor of protein tyrosine kinases. Akiyama, T.; Ishida, J.; Nakagawa, S.; Ogawara, H.; Watanabe, S.; Itoh, N. M.; Shibuya, M.; Fukagami, Y. “Genistein, a specific inhibitor of tyrosine-specific protein kinase” J. Biol. Chem., 262: 5592-5595 (1987). Many cellular biochemical pathways, initiated by extracellular growth factors, proceed by the phosphorylation of tyrosine using protein tyrosine kinases. These pathways are especially important in transformed cells, since more than half of the protein products of cellular oncogenes undergo uncontrolled tyrosine phosphorylation. Cantley, L. C.; Auger, C.; Carpenter, B.; Duckworth, R.; Kapeller, R.; Soltoff, S. “Oncogenes and signal transduction” Cell, 64: 281-302 (1991). In light of these investigations, genistein has been a subject of numerous studies involving protein tyrosine kinase inhibition. Genistein inhibits the proliferative growth of many cancer cells in tissue culture, inhibits the appearance of tumors in animal models of breast cancer and skin cancer, and is active against colonic cancer. Peterson, T. G.; Barnes, S. “Genistein inhibition of the growth of human breast cancer cells: independence from estrogen receptors and the multi-drug resistance gene” Biochem. Biophys. Res. Commun., 179: 661-667 (1991); Peterson, T. G.; Barnes, S. “Genistein potently inhibits the growth of human primary breast epithelial cells: correlation with lack of genistein metabolism” Mol. Biol. Cell, 5: 384a (1994); Lamartiniere, C. A.; Moore, J. A.; Holland, M.; Barnes, S. “Genistein and chemoprevention of breast cancer” Proc. Soc. Exptl. Biol. Med., 208: 120-123 (1995); Wei, H.; Wei, L.; Frankel, K.; Bowen, R.; Barnes, S. “Inhibition of tumor promoter-induced hydrogen peroxide formation in vitro and in vivo by genistein” Nutr. Cancer, 20: 1-12 (1993); Wei, H.; Bowen, R.; Cai, Q.; Barnes, S.; Wang, Y. “Antioxidant and antipromotional effects of the soybean isoflavone genistein” Proc. Soc. Exptl. Biol. Med., 208: 124-130 (1995); Pereira, M. A.; Barnes, L. H.; Rassman, V. L.; Kelloff, G. V.; Steele, V. E. “Use of azoxymethane-induced foci of aberrant crypts in rat colon to identify potential cancer chemopreventative agents” Carcinogenesis, 15: 1049-1054 (1994); Helms, J. R.; Gallaher, J. J. “The effect of dietary soy protein isolate and genistein on the development of preneoplastic lesions (aberrant crypts) in rats” J. Nutr., 125: 802S (1995). Apart from the anti-cancer applications, genistein is also a good inhibitor of alpha-glucosidase (invertase). Lee, D. H.; Lee, S. H. “Genistein, a soy isoflavone, is a potent alpha-glucosidase inhibitor” FEBS Lett., 501(1); 84-86 (2001). Genistein is a reversible, slow-binding, non-competitive inhibitor of alpha-glucosidase with a Ki value of 5.7×10⁻⁸ M. These results indicate that genistein can be used to treat certain metabolic disorders. Isoflavones are also known to lower the incidence of coronary heart disease and mitigate the effects of hot flashes in postmenopausal women. In many cases, treatment of hot flashes by isoflavones has proven safer and more effective than estrogen replacement therapy.

### SUMMARY OF THE INVENTION

The present invention is directed to a composition and method increasing the bioavailability of one or more isoflavonoids and plant polyphenols in a subject. The composition comprises one or more enzymes selected from the group consisting of xylanase, beta-glucanase and beta-glucosidase and mixtures thereof. The composition may be formulated into various pharmaceutical dosage forms such as tablets, capsules, chewable tablets, powders, effervescent tablets or powders, solutions, suspensions, or emulsions suitable for oral, nasogastric or other enteral administration.

The method comprises co-administering the inventive composition to the subject with oral glycosylated isoflavones to cause the in vivo cleavage of the beta-glycosidic linkages resulting in bioavailable aglycones. One such composition is a combination of xylanase and beta-glucanase called Isolase™ by Applicant, which is described in more detail herein.

### BRIEF DESCRIPTION OF THE DRAWINGS

FIG. 1 is a line graph with the percent of soy isoflavone genistin hydrolyzed into genistein by the combination of xylanase and glucanase in 15 minutes with the percent of hydrolysis on the Y axis and the amount in milligrams of the combination of xylanase and glucanase (Isolase™) on the X axis.

FIG. 2 is a line graph with the percent of soy isoflavone genistin hydrolyzed into genistein by the combination of xylanase and glucanase in 30 minutes with the percent of hydrolysis on the Y axis and the amount in milligrams of the combination of xylanase and glucanase (Isolase™) on the X axis.

FIG. 3 is a line graph with the percent of soy isoflavone genistin hydrolyzed in vitro into genistein by the combination of xylanase and glucanase in 45 minutes with the percent of hydrolysis on the Y axis and the amount in milligrams of the combination of xylanase and glucanase (Isolase™) on the X axis.

FIG. 4 is a line graph with the percent of soy isoflavone genistin hydrolyzed in vitro into genistein by the combination of xylanase and glucanase in 60 minutes with the percent of hydrolysis on the Y axis and the amount in milligrams of the combination of xylanase and glucanase (Isolase™) on the X axis.

FIG. 5 is a line graph with the percent hydrolysis in vitro of genistin into genistein from 100 mg of soy isoflavone with 5 mg of the combination of xylanase and glucanase (Isolase™). The percent of hydrolysis is on the Y axis, and the minutes incubated is on the X axis.

FIG. 6 is a line graph with the percent hydrolysis in vitro of genistin into genistein from 100 mg of soy isoflavone with 12 mg of the combination of xylanase and glucanase (Isolase™). The percent of hydrolysis is on the Y axis, and the minutes incubated is on the X axis.

FIG. 7 is a line graph with the percent hydrolysis in vitro of genistin into genistein from 100 mg of soy isoflavone with 18 mg of the combination of xylanase and glucanase (Isolase™). The percent of hydrolysis is on the Y axis, and the minutes incubated is on the X axis.

FIG. 8 is a line graph with the percent hydrolysis in vitro of genistin into genistein from 100 mg of soy isoflavone genistin into genistein with 25 mg of the combination of xylanase and glucanase (Isolase™). The percent of hydrolysis is on the Y axis, and the minutes incubated is on the X axis.

FIG. 9 is a line graph with the percent hydrolysis in vitro of genistin into genistein from 100 mg of soy isoflavone genistin into genistein with 30 mg of the combination of xylanase and glucanase (Isolase™). The percent of hydrolysis is on the Y axis, and the minutes incubated is on the X axis.

FIG. 10 is a bar graph illustrating the absorption of genistein with and without the administration of the inventive combination of xylanase and glucanase (Isolase™). The bar graph represents the amount of genistein present in human plasma three (3) hours after ingestion of Isolase™.

FIG. 11 is a bar graph illustrating the absorption of daidzein with and without the administration of the inventive combination of xylanase and glucanase (Isolase™). The bar graph represents the amount of daidzein present in human plasma three (3) hours after ingestion of Isolase™.

FIG. 12 is a line graph illustrating the pH profile of one embodiment of the present invention, specifically the combination of xylanase and glucanase (Isolase™). The percent activity is on the Y axis and the pH is on the X axis.

FIG. 13 is a bar graph illustrating the temperature profile of one embodiment of the present invention, specifically the combination of xylanase and glucanase (Isolase™). The percent activity is on the Y axis and the temperature is on the X axis.

FIG. 14 is a bar graph illustrating the stability of one embodiment of the present invention (Isolase™) in an environment that mimics the gut of a human. The activity of Isolase™ versus a control in IsoU/gram units is on the Y axis and the time is on the X axis.

### DETAILED DESCRIPTION OF THE INVENTION

While the present invention may be embodied in many different forms, several specific embodiments are discussed herein with the understanding that the present disclosure is to be considered only as an exemplification of the principles of the invention and is not intended to limit the invention to the embodiments illustrated.

In one embodiment of the present invention, the enzyme(s) used to cleave the glycosylated isoflavone to the aglycone form cleave the beta-glycosidic linkage that is the dominant linkage between the isoflavone and its carbohydrate (normally glucose) moiety. The inventors unexpectedly found that a xylanase/glucanase enzyme mix is highly effective in cleaving the glycosylated isoflavone to the aglycone form.

Another embodiment of the present invention is a method for converting a glycosylated isoflavone into an aglycone in a digestive tract of a subject in need thereof. The method comprises orally administering to the subject an effective amount of a composition comprising at least one enzyme, for example, a xylanase, a glucanase, a alpha-galactosidase, a lactase or a glucosidase; and concomitantly administering to the subject a food stuff, for example glycolsylated isoflavone. In another embodiment of the present invention the xylanase is endo-1,4-beta-xylanase and the glucanase is beta-glucanase. In yet another embodiment of the present invention the composition comprises about two parts endo-1,4-beta-xylanase to about one part beta-glucanase. It is further contemplated that the endo-1,4-beta-xylanase may be isolated from T. longibrachiatum and the beta-glucanase may be isolated from A. niger.

In another illustrative embodiment of the present invention the aglycone comprises genistein, formononetin, benistein, biochanin A, daidzein, or glycetein.

In another embodiment of the present invention, the invention comprises ingesting the composition concurrently or sequentially with the oral ingestion of a glycoside. In one embodiment of the present invention, the glycoside is a flavonoid. The flavonoid can further be an isoflavone, for example.

In another embodiment the isoflavone of the present invention may be isolated from a source such as, for example, black cohosh, red clover or soy.

In one embodiment of the present invention, the composition and method comprises at least one protease. In yet another embodiment of the present invention, the protease comprises pepsin, papain, trypsin, collagenase, liberase, and a proteolytic enzyme from a plant, an animal, a fungal or a microbial source.

In another embodiment of the present invention, the composition and method comprises hemicellulase and/or a probiotic.

In one embodiment of the present invention, the invention is a pharmaceutical dosage form, comprising about 5 mg to about 500 mg of a composition comprising a xylanase and a glucanase. In another embodiment of the present invention, the dosage form comprises about two parts xylanase to about one part glucanase. In yet another embodiment of the present invention, the dosage form comprises a tablet, soft capsule and hard gelatin capsule, suspension tablet, effervescent tablet, powder, effervescent powder, chewable tablet, solution, suspension, or an emulsion.

Also provided is a method for increasing a serum concentration of an aglycone in a subject in need thereof. The method comprises administering to the subject a pharmaceutical dosage form comprising about 5 mg to about 500 mg of a composition comprising a xylanase and a glucanase. In yet another embodiment of the present invention, the method further comprises ingesting the composition concurrently with oral ingestion of a glycoside. In another embodiment, the xylanase is endo-1,4-beta-xylanase and the glucanase is beta-glucanase. In another embodiment of the present invention the composition comprises about two parts endo-1,4-beta-xylanase to about one part beta-glucanase.

Another embodiment of the present invention is a method for converting genistin into genistein in a digestive tract of a subject in need thereof. The method comprises orally administering to the subject about 5 mg to about 500 mg of a composition comprising endo-1,4-beta-xylanase and beta-glucanase concurrently or sequentially with the ingestion of a soy food stuff.

Another illustrative embodiment of the present invention is a method for converting genistin into genistein in a digestive tract of a subject in need thereof. The method comprises orally administering to the subject about 5 mg to about 500 mg of a composition comprising about two parts endo-1,4-beta-xylanase and about one part beta-glucanase concurrently or sequentially with the ingestion of a soy food stuff.

Another embodiment of the present invention is a pharmaceutical dosage form, comprising about 5 mg to about 500 mg of a composition comprising about two parts endo-1,4-beta-xylanase and one part beta-glucanase.

Yet another embodiment of the present invention is a method for increasing a serum concentration of an aglycone in a subject in need thereof. The method comprises administering to the subject a pharmaceutical dosage form comprising about 5 mg to about 500 mg of a composition comprising about two parts endo-1,4-beta-xylanase and about one part beta-glucanase per dose.

Another embodiment of the present invention is a method for increasing a serum concentration of genistein in a subject in need thereof. The method comprises administering to the subject a pharmaceutical dosage form comprising about 5 mg to about 500 mg of a composition comprising about two parts endo-1,4-beta-xylanase and about one part beta-glucanase.

In another embodiment of the present invention, the pharmaceutical dosage form is produced according to the process of dry granulating, wet granulating, or dry mixing endo-1,4-beta-xylanase, beta-glucanase, and an optional pharmaceutically acceptable excipient; and forming the endo-1,4-beta-xylanase, beta-glucanase, and the optional pharmaceutically acceptable excipient into a tablet, soft gelatin capsule, hard gelatin capsule, suspension tablet, effervescent tablet, powder, effervescent powder, and chewable tablet.

Yet another embodiment of the present invention is a process of manufacture of a pharmaceutical dosage form comprising dry granulating, wet granulating, or dry mixing endo-1,4-beta-xylanase, beta-glucanase, and optional pharmaceutically acceptable excipient; and forming the endo-1,4-beta-xylanase, beta-glucanase, and the optional pharmaceutically acceptable excipient into a tablet, soft gelatin capsule, hard gelatin capsule, suspension tablet, effervescent tablet, powder, effervescent powder, and chewable tablet.

The term “subject” as used herein, includes, for example, mammals, reptiles, birds, exotic animals and farm animals, including rodents, and the like. In one embodiment, the mammal includes a primate, for example, a human, a monkey, a horse, a dog, a pig, or a cat. In another embodiment, the rodent includes a rat, a mouse, a squirrel or a guinea pig.

The term “isoflavone” as used herein, describes a non-carbohydrate plant constituent. In nature, isoflavones can be found in a variety of forms. The glycosylated form is the most common form. One explanation of the form preference is that the glycoside form is water-soluble and also provides enhanced stability to degradative factors such as heat, oxidation and ultraviolet irradiation.

One particularly good source of isoflavones is soybeans. Isoflavones found in soybeans typically are found in both the glycosylated and aglycone form; however, the glycosylated forms dominate. The glycosylated forms include genistin, daidzin and glycitin. The aglycone forms include genistein, daidzein and glycitein respectively. In one embodiment of the present invention the glycosylated forms of the isoflavones is converted in vivo to the more bioavailable aglyconated forms, and thus increases the serum concentrations of such forms in a subject.

As used herein, the term “effective amount,” means the dose of enzyme(s) of the present invention, which results in a therapeutic level of one or more aglycones (“therapeutic agent”) in the bloodstream of the subject after the concomitant oral administration of the enzyme composition and glycone source material. It is understood, however, that the effective amount of the inventive composition and the resulting levels of the therapeutic agents for any particular patient depend upon a variety of factors, including the activity of the specific enzyme(s) employed, as well as the age, body weight, general health, sex and diet of the subject. Dose-effect relationships from in vitro and/or in vivo tests initially can provide useful guidance on the proper doses for subject administration. Studies in animal models generally may be used for guidance regarding effective doses in accordance with the present invention. In terms of treatment protocols, it should be appreciated that the dose to be administered will depend on several factors, including the particular enzyme and glycosylated isoflavones that are administered, the condition of the particular subject, etc. Generally speaking, one will desire to administer the enzyme combination and glycosylated isoflavones to produce a bioavailable amount of the therapeutic agent that is effective to elicit a therapeutic effect.

In another embodiment of the present invention, the time of administration of an effective amount of the enzyme combination of the invention is concomitant with an isoflavone foodstuff. “Concomitantly,” or “concomitant,” or “concurrently” or “concurrent” as used herein, is the oral or enteral administration of the enzyme composition and the glycone (e.g., isoflavone) source material in a substantially simultaneous manner in a single formulation or in separate formulations. Substantially simultaneous administration can be accomplished, for example, by administering to the subject a single capsule, tablet or solution or suspension having a fixed ratio of enzyme(s), or in multiple, single capsules, tablets, or solutions for each of the enzymes, together with the glycone source material. In one embodiment of the present invention, concurrently or concomitantly includes, for example, administration of the enzyme combination and the food stuff occurs within several seconds, for example, within ten to sixty seconds; or within minutes, for example, within one to sixty minutes; or within hours, for example, one to twenty four; such that a beneficial effect from the co-action of these agents is achieved.

The enzyme composition and the glycone (e.g. isoflavone) source material of the present invention may also be administered sequentially.

To determine the effectiveness of a particular enzyme an “Enzyme Effectiveness Analysis” was performed by Applicants that measured the activity of a particular enzyme or enzyme combination. The analysis was used to determine the activity and effectiveness of an enzyme's ability to cleave the β-D-glucopyranosidic bond. The assay utilized the hydrolysable substrate p-nitrophenyl-β-D-glucopyranoside. After the particular enzyme or enzyme combination was incubated with the substrate and it cleaved the β-D-glucopyranosidic bond, it liberated the p-nitrophenol component. The hydrosylate, p-nitrophenol, linearly absorbs light at 405 nm. Thus, depending on the absorption measured by a spectrophotometer, it was determined how much p-nitrophenol was liberated. One unit (“U”) was assigned to the amount of an enzyme that is required to liberate p-nitrophenol at a rate of 0.01 micromole per minute. Thus, each enzyme's ability to cleave the β-D-glucopyranosidic bond can be described in these particular units. For example, if the particular enzyme were beta-glucanase (BG), the activity can be described as BGU/gram.

In one embodiment of the present invention, the enzyme combination is two parts endo-1,4-beta-xylanase (T. longibrachiatum) and one part beta-glucanase (A. niger). Using the above described procedure, a unit of measure was determined for this embodiment of the present invention. This particular combination was named Isolase™, thus, IsoU is used as unit of measure. The activity for this particular enzyme combination was calculated as follows:

IsoU/g=(ΔE×V×D×10⁶×100)/(Δt×E×c)

where: ΔE=change in absorbance @ 405 nm, from 2 to 10 minutes.

V=Total volume in the cuvette (2.1 ml)

E=mMolar extinction coefficient of p-nitrophenyl @ 405 nm (use 13,000)

D=Dilution factor (always 1 when enzyme stock solution is used without further dilution)

Δt=elapsed reaction time between measurements (8 minutes).

c=Weight of Isolase™ in grams used to prepare enzyme stock solution.

10⁶=Converts mole to μmole

100=Constant

### EXAMPLES

Several experiments were performed in accordance to the above-described Enzyme Effectiveness Analysis. In the experiments, 0, 5, 12, 18, 25, and 30 mgs of enzyme combination of two parts xylanase and one part beta-glucanase was incubated for a period of 15 to 60 minutes with the 40% soy isoflavone substrate, NEC #21292001 (National Enzyme Company, 15366 U.S. Highway 160, Forsyth, Mo. 65653). The soy isoflavone substrate contained about 350 mg/g of genistin, about 70 mg/g of daidzin, and about 4 mg/g of glycetin. A standard curve was determined for genistein and a response factor was applied to measure the extent of total soy isoflavone hydrolysis that was achieved with each test.

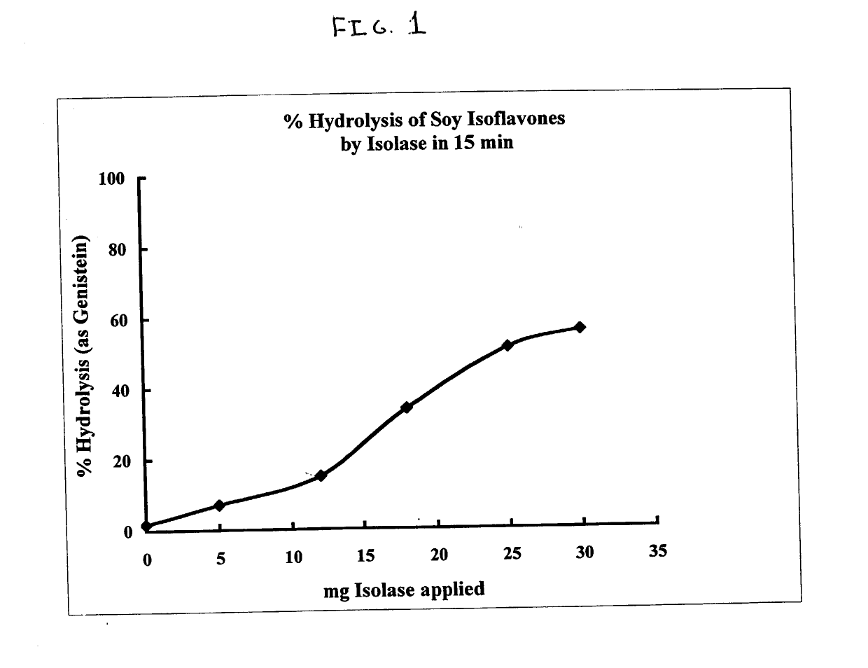

In a first experiment, the enzyme combination was incubated with the soy substrate NEC #21292001 for fifteen (15) minutes. The amount of enzyme combination was varied form zero (0) to thirty (30) mg. As shown in FIG. 1, a dose of about thirty (30) milligrams and an incubation time of fifteen minutes the enzyme combination hydrolyzed about fifty-five percent (55%) of the isoflavone, genistin into its aglycone form, genistein.

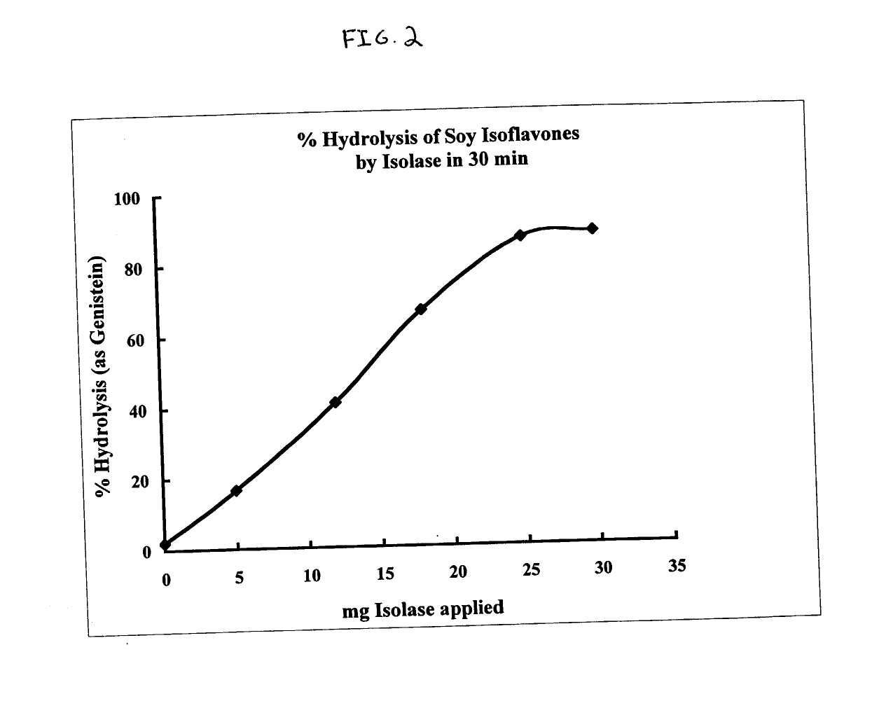

In a second experiment, the enzyme combination was incubated with the soy substrate NEC # 21292001 for thirty (30) minutes and the amount of enzyme combination was varied from about zero (0) to about thirty (30) mg. As shown in FIG. 2, a dose of about thirty (30) milligrams and an incubation of thirty minutes resulted in about eighty-eight percent (88%) hydrolysis of the isoflavone, genistin into its aglycone form, genistein.

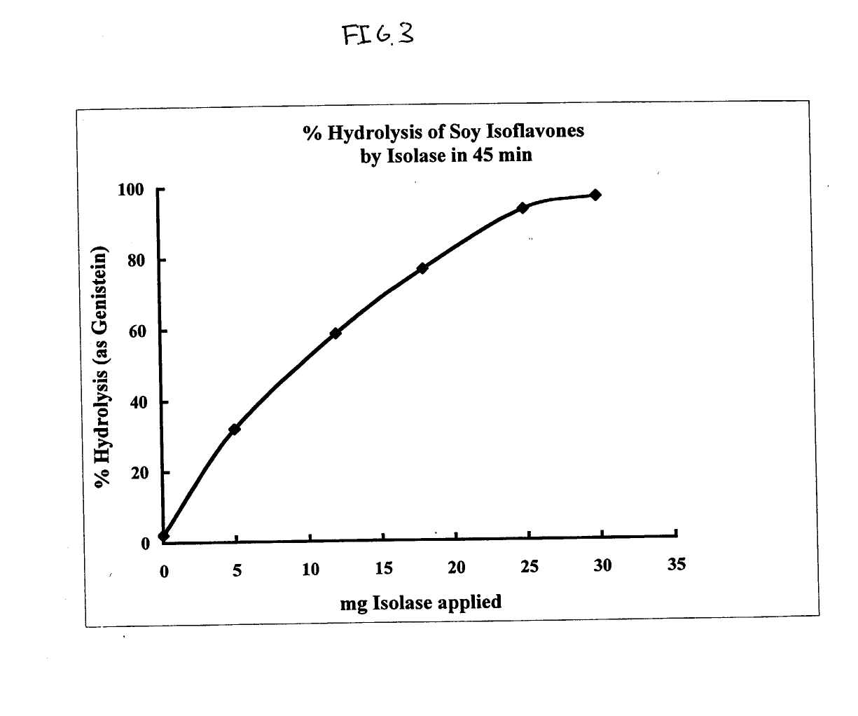

In a third experiment, the enzyme combination was incubated with the soy substrate NEC #21292001 for forty-five (45) minutes and the amount of enzyme combination was varied from about zero (0) to about thirty (30) mg. As shown in FIG. 3, a dose of about thirty (30) milligrams and an incubation of forty-five (45) minutes resulted in about ninety-seven percent (97%) hydrolysis of the isoflavone, genistin into its aglycone form, genistein.

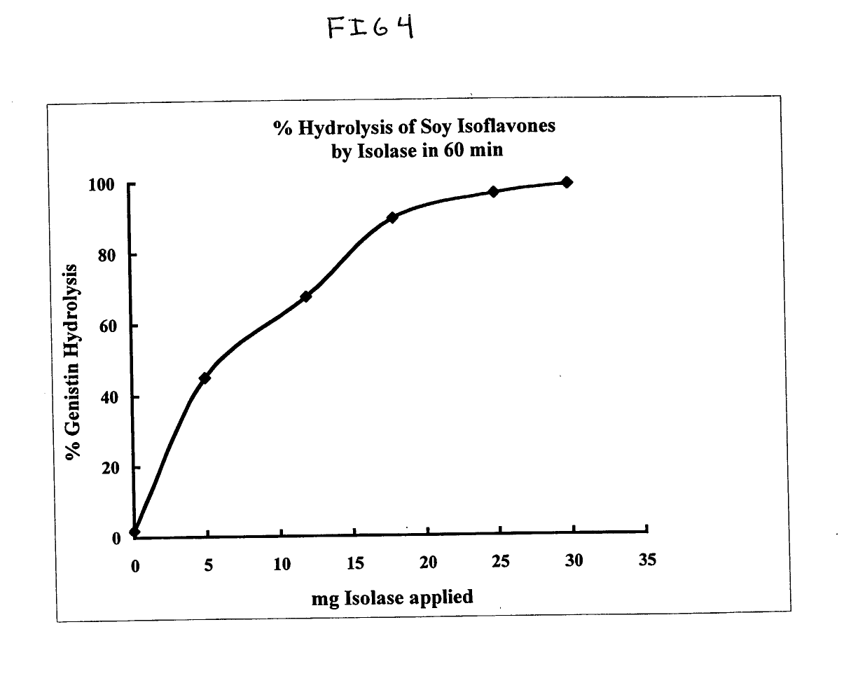

In a fourth experiment, the enzyme combination was incubated with the soy substrate NEC #21292001 for sixty (60) minutes and the amount of enzyme combination was varied from about zero (0) to about thirty (30) mg. As shown in FIG. 4, a dose of about thirty (30) milligrams and an incubation of sixty (60) minutes resulted in about ninety-nine percent (99%) hydrolysis of the isoflavone, genistin into its aglycone form, genistein.

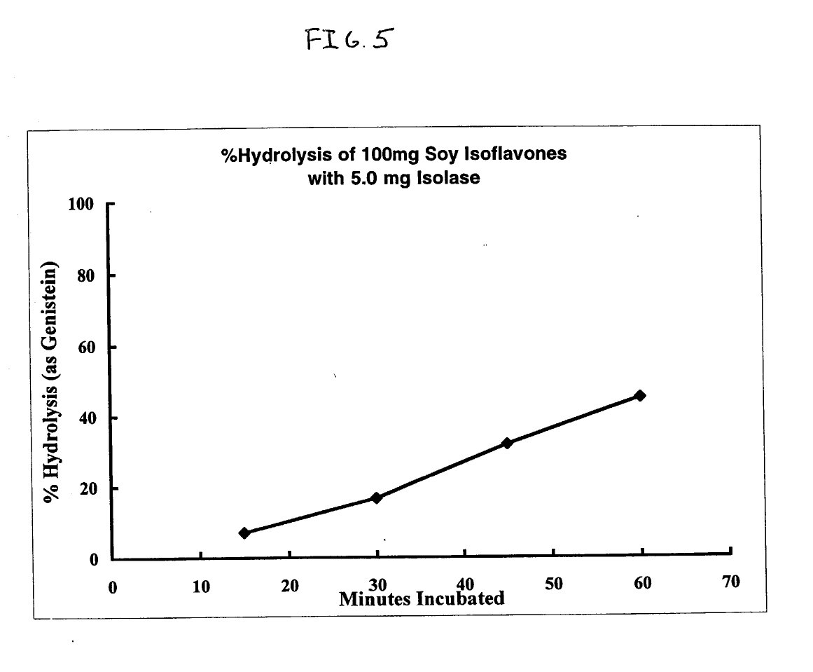

In a fifth experiment, one hundred (100) milligrams of the soy isoflavone substrate NEC #21292001 was incubated with five (5) milligrams of the enzyme combination and the percent hydrolysis of genistin into the aglycone form, genistein was measured. As shown in FIG. 5, after about thirty (30) minutes 18 percent (18%) was hydrolyzed and after about sixty (60) minutes forty-five percent was hydrolyzed.

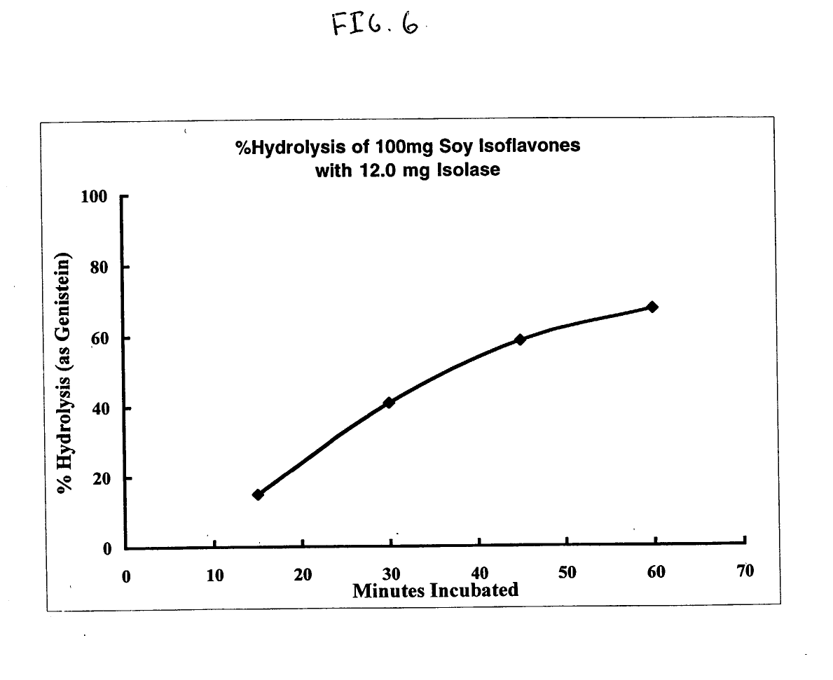

In a sixth experiment, one hundred (100) milligrams of the soy isoflavone substrate NEC #21292001 was incubated with twelve (12) milligrams of the enzyme combination and the percent hydrolysis of genistin into the aglycone form, genistein was measured. As shown in FIG. 6, after about thirty (30) minutes forty percent (40%) was hydrolyzed and after about sixty (60) minutes seventy percent (70%) was hydrolyzed.

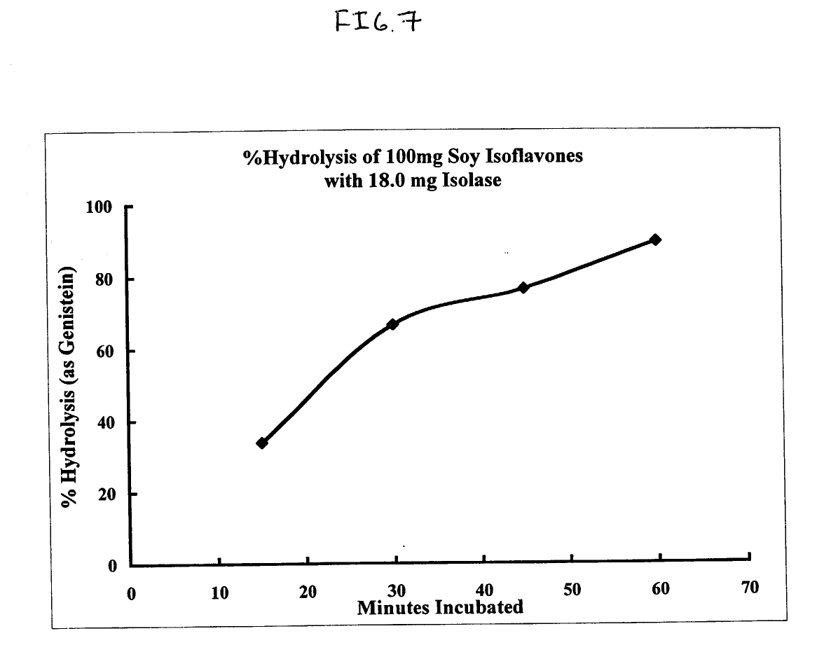

In a seventh experiment, one hundred (100) milligrams of the soy isoflavone substrate NEC #21292001 was incubated with eighteen (18) milligrams of the enzyme combination and the percent hydrolysis of genistin into the aglycone form, genistein was measured. As shown in FIG. 7, that after about thirty (30) minutes sixty-five percent (65%) was hydrolyzed and after about sixty (60) minutes ninety percent (90%) was hydrolyzed.

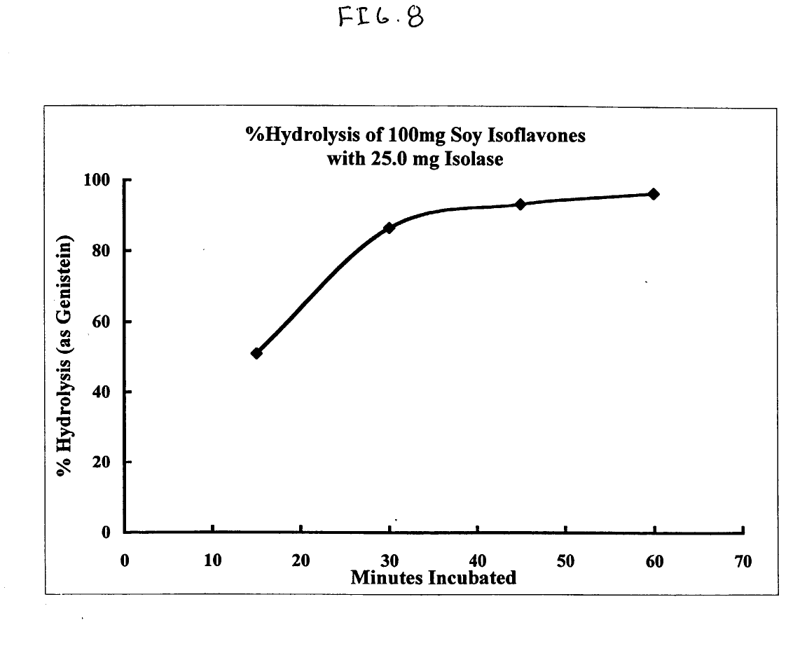

In an eighth experiment, one hundred (100) milligrams of the soy isoflavone substrate was incubated with twenty-five (25) milligrams of the enzyme combination and the percent hydrolysis of genistin into the aglycone form, genistein was measured. As shown in FIG. 8, after about thirty (30) minutes eighty percent (85%) was hydrolyzed and after about sixty (60) minutes ninety-eight percent (98%) was hydrolyzed.

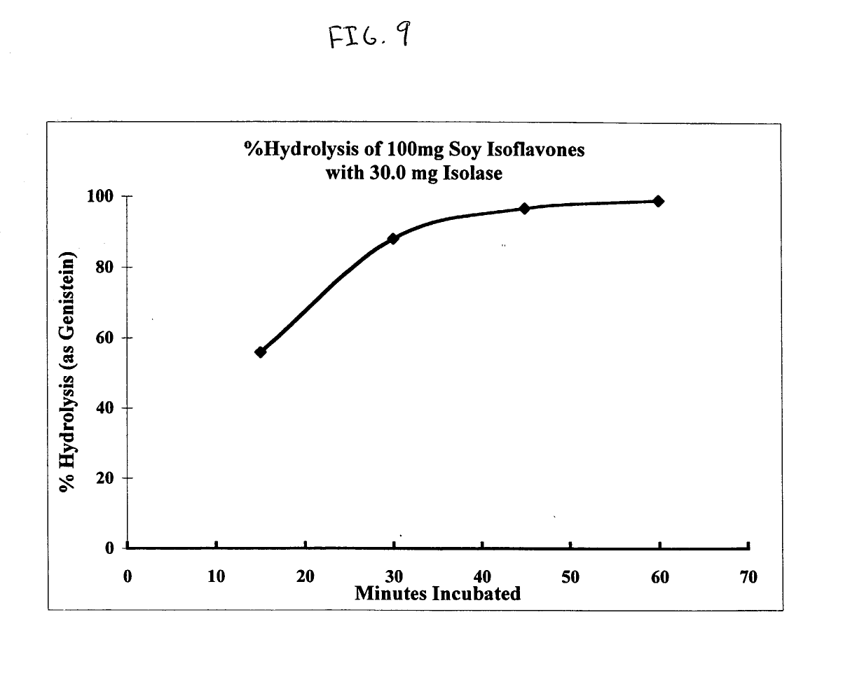

In a ninth experiment, one hundred (100) milligrams of the soy isoflavone substrate was incubated with thirty (30) milligrams of the enzyme combination and the percent hydrolysis of genistin into the aglycone form, genistein was measured. As shown in FIG. 9, that after about thirty (30) minutes ninety percent (90%) was hydrolyzed and after about sixty (60) minutes ninety-nine percent (99%) was hydrolyzed.

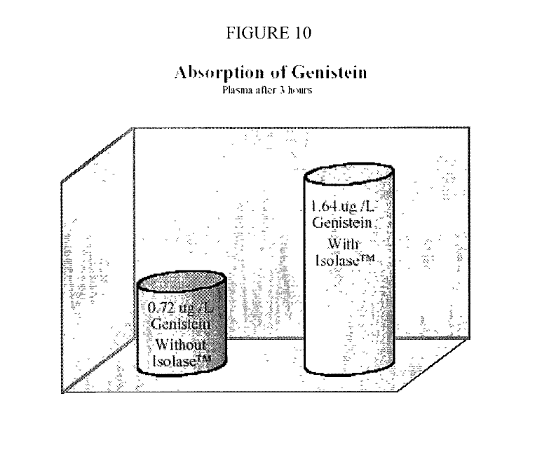

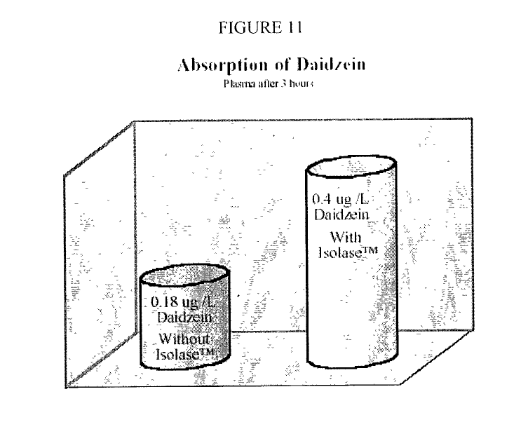

In another experiment, the plasma concentration and urine levels of aglycone isoflavones were measured in humans. The subjects were separated using a randomized cross-over design. The subjects were administered a dose of about four (4) grams of a soy milk powder, which was combined with about 300 milligrams of an enzyme mixture in an eight ounce beaker. The enzyme mixture comprised about 1200 IsoU or about 10,000 XU (xylanase) combined with 554 BGU (beta-glucanase). Just prior to the subject ingesting the soy milk powder and enzyme mixture it was reconstituted with water, then immediately thereafter consumed by the subject. The subjects collected all urine for twenty-four (24) hours, plus the first urination on the morning after isoflavone dosing. Blood was taken at zero (0) hours, three (3) hours, and twenty-four (24) hours post-dosing. The amount of aglycone present in the serum was then measured. The results of this experiment (FIGS. 10 and 11) demonstrated the effectiveness of the invention to increase the bioavailability of the aglycones in human subjects.

Illustratively, using the above information, the xylanase/glucanase enzyme combination (i.e., 10,000 XU and 554 BGU), for example, can be used concomitantly with one or more glycone sources as follows:

| | Glycosylated | Approximate Milligrams |
|---|---|---|
| Isoflavone Source | Isoflavones Present | of Enzyme Suggested |
| 10 g Soy Flour | 161 mg Isoflavones | 56 mg Enzyme Composition |
| 10 g Soy Protein | 100 mg Isoflavones | 35 mg Enzyme Composition |
| Conc. | | |
| 10 g Soy Protein | 21 mg Isoflavones | 7.35 mg Enzyme Composition |
| Isolate | | |
| 10 g Soy Sprouts | 136 mg Isoflavones | 48 mg Enzyme Composition |
| 100 g Instant Soy | 110 mg Isoflavones | 39 mg Enzyme Composition |
| Beverage | | |
| 100 g Miso | 43 mg Isoflavones | 15 mg Enzyme Composition |
| 100 mg Tofu | 25 mg Isoflavones | 9 mg Enzyme Composition |

Any enzyme employed to achieve the desired results is contemplated by the present invention. For example, the following enzymes may be employed, either singly or in combination: (1) xylanase (T. longibrachiatum) enzyme can be used from a standardized preparation with an activity range of 1,000 XU's/gram to 100,000 XU/gram; (2) beta-glucanase (Aspergillus niger) enzyme can be used from a standardized preparation with an activity range of 350 BGU/gram to 35,000 BGU's/gram; (3) beta-glucosidase (Aspergillus niger) enzyme can be used from a standardized preparation with an activity range of 100 IsoU/gram to 100,000 IsoU/gram; (4) alpha-galactosidase (Aspergillus niger) enzyme can be used from a standardized preparation with an activity range of 500 GalU/gram to 100,000 GalU/gram; (5) protease (Aspergillus oryzae, Aspergillus niger, Bacillus subtilis, Ananas cosmosus, or Carica papaya) enzyme can be used from a standardized preparation with an activity range of 1,000 HUT/gram to 600,000 HUT/gram; (6) lactase (Aspergillus niger) enzyme can be used from a standardized preparation with an activity range of 100 ALU/gram to 100,000 ALU/gram; and (7) probiotic preparations (Bifidobacterium longum, Lactobacillus acidophilus, Lactobacillus casei, Lactobacillus planatarum, Enterococcus faecium, Bifidobacterium bifidum, Lactobacillus salivarius, Lactobacillus rhamnosis, Lactobacillus reuteri, Lactobacillus bulgaricus, Streptococcus thermophilus, Bifidobacterium infantis, Bifidobacterium breve, or Lactobacillus sporogenes) can be used from a standardized preparation with an active culture range of 1 million to 100 billion colony-forming units (CFU)/gram. Variations in dose and content may be made to tailor enzymatic supplementation to a specific substrate.

The enzyme composition may also be combined with a protease. The protease is advantageously included to degrade certain plant material. Examples of a protease that may be combined with the enzyme composition of the present invention are pepsin, papain, trypsin, collagenase, liberase, and other proteolytic enzymes.

It is further contemplated that the enzyme combination may be combined with hemicellulase. The hemicellulase activity is deemed to be beneficial by degrading hemicellulosic constituents contained in certain plant material. Further, the present invention can be combined with CereCalase™ (National Enzyme Company) additive for red clover or black cohosh applications, where interferences may be expected in the form of plant cellular matrices. Additionally, the present invention can be combined with other active agents, such as antioxidants, vitamins, or other pharmaceutical agents.

The materials that may be used to supply the source of isoflavones include any material that contains detectable levels of isoflavones, not only soybeans. Illustratively, plant isoflavones are obtainable from many herbal/botanical preparations that may include (but are not limited to) alfalfa grass, barley grass, buckwheat, echinacea, ginkgo, ginseng, golden seal, gotu kola, green tea, hops flowers, cayenne fruit, valerian root, etc. Isoflavones may also be synthesized.

In one embodiment of the present invention, the composition containing the enzymes and the glycosylated isoflavones, are administered in the same dosage form or concomitantly. The doses for each component can be measured separately and can be given as a single combined dose or given separately. They may be given at the same or at different times as long as the enzymes and glycosylated isoflavones are in the gastrointestinal tract patient concurrently. Concomitant or concurrent administration means the patient ingests at least one enzyme and the isoflavone such that a beneficial effect from the co-action of these agents is achieved. Because the goal is to provide increased polyphenol bioavailability to the patient, in most cases when the enzyme(s) is ingested the remaining enzyme and/or glycosylated isoflavone would be administered to the patient close in time and typically concomitantly. Order of administration is not important.

Illustratively, the administration of an effective amount of the enzyme combination of the present invention concomitantly with an isoflavone source increases the bioavailability of the aglycones and subsequently increase the serum level of the aglycones. The method of raising the serum level of aglycones to treat disease states is contemplated in the present invention. Examples of disorders in which the present invention contemplates treatment are, for example, breast cancer, colonic cancer, skin cancer, post-menopausal symptoms such as hot flashes, proliferative growth of other forms of cancer cells, heart disease and metabolic disorders.

It is contemplated in the present invention that the methods described herein are applicable to all forms of glycosides. All glycosides have a similar glycosylated form in which a non-carbohydrate molecule has a beta-glycosidic linkage to a sugar. The linkage is equally susceptible to the present invention. Glycosides include, but are not limited to, phenols and polyphenols, flavonoids, lactones, aldehydes, anthraquinones, saponins, etc.

The desirability of the present invention is demonstrated by a recent study using rats and analyzing the polyphenol bioavailability. Keiko Azuma et al., “Absorption of Chlorogenic Acid and Caffeic Acid in Rats after Oral Administration,” J. Agric. Food Chem. 2000, 48 pp. 5496-5500. Rats were administered 700 μM/kg body weight of chlorogenic or caffeic acid, and blood was collected from the tail for 6 hours after administration. The ingested chlorogenic acid (gluconated polyphenol) was not readily absorbed, and its various conjugates were present at maximum serum levels in one hour, in the amount of 0.34 μM. The ingested caffeic acid (aglycone polyphenol) was more readily absorbed, and its various conjugates were present at maximum serum levels in 2 hours at a concentration level of 0.64 μM. This represented a 188 times increase in absorption.

Additionally, the bioavailability of flavonoids was discussed in another recent article. Crepsy, V et al., “Quercetin, but not its Glycosides, is Absorbed from the Rat Stomach,” J. Agric. Food Chem., 2002, 50, pp. 618-621. Fifteen (15) μM of quercetin, isoquercitrin, or rutin were administered in the stomachs of rats in situ, and after 30 minutes the stomach contents, bile and serum levels were investigated for levels of the previously-mentioned polyphenols and their metabolites. The administration of quercetin (aglycone flavonoid) resulted in 4.1 μM found in the bile samples and 8.6 μM remaining in the stomach after 30 minutes. Conversely, after 30 minutes the entire dose of glycosides (15 μM rutin and 15 μM isoquercitrin) remained in the stomach.

The pharmacokinetics of daidzein and genistein in adults according to a recent study has established that the volume of distribution in adults is large, indicating a wide tissue distribution, and that the shape of plasma appearance curve is consistent with compounds that undergo enterohepatic recycling. Setchell K D, “Absorption and Metabolism of Soy Isoflavones from Food to Dietary Supplements and Adults to Infants” Journal of Nutrition, 130: 645-655 (2000). The Study further found, peak concentrations are seen generally 4-8 hours after dietary intake, and the plasma appearance and disappearance in pre- and postmenopausal women are similar. Most of the daidzein and genistein is excreted within the first 24 hours with the average fractional excretion remaining relatively constant over a wide range of intakes, although there is a high individual variability, ranging from 20 to 50% of the dietary intake. Differences are observed in the elimination half-life for different foods. More rapid elimination is observed for isoflavones in a liquid matrix than in a solid matrix food. A curvilinear relationship was found between the dietary intake of isoflavones and peak plasma concentration and apparent bioavailability, indicating that absorption of isoflavones from food may be saturable. It may be more difficult to attain supraphysiologic levels of isoflavones from foods than from supplements, which are more closely aligned with pharmacologic agents. On the basis of the pharmacokinetics of soy isoflavones, maintenance of high steady-state plasma concentrations will be achieved by a regular intake, particularly if phytoestrogens are ingested throughout the day.

The enzymes of the present invention are usually administered in the form of pharmaceutical compositions. These enzyme agents can be administered by a variety of oral routes, as described below. When administered, the enzymes of the present invention are administered in pharmaceutically acceptable compositions. Such preparations may routinely contain buffering agents, preservatives, compatible carriers and other therapeutic ingredients.

The present invention also includes methods employing pharmaceutical compositions that contain the enzymes of the present invention associated with pharmaceutically acceptable carriers or excipients. As used herein, the terms “pharmaceutically acceptable carrier” or “pharmaceutically acceptable excipients” includes any and all solvents, dispersion media, coatings, antibacterial and antifungal agents, isotonic and absorption delaying agents, and the like. The use of such media and agents for ingestible substances is well known in the art. Except insofar as any conventional media or agent is incompatible with the enzymes, its use is contemplated. Supplementary active ingredients can also be incorporated into the compositions.

In making the compositions of the present invention, the enzyme(s) can be mixed with a pharmaceutically acceptable excipient, diluted by the excipient or enclosed within such a carrier, which can be in the form of a capsule, sachet, paper or other container. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents, such as talc, magnesium stearate and mineral oil; wetting agents; emulsifying and suspending agents; preserving agents, such as methyl- and propylhydroxybenzoates; sweetening agents; and flavoring agents. The compositions of the present invention can be formulated so as to provide quick, sustained or delayed release of the enzymes after administration to the patient by employing procedures known in the art.

When the excipient serves as a diluent, it can be a solid, semi-solid or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), soft and hard gelatin capsules and sterile packaged powders.

Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents and pharmaceutically compatible carriers. In one embodiment of the present invention, the manufacturing processes may employ one or a combination of four established methods: (1) dry mixing, (2) direct compression, (3) milling, and (4) non-aqueous granulation. Lachman et al., The Theory and Practice of Industrial Pharmacy (1986). Such tablets may also comprise film coatings, which dissolve upon oral ingestion or upon contact with diluent.

In another embodiment of the present invention, solid compositions, such as tablets, are prepared by mixing the enzymes with a pharmaceutical excipient to form a solid preformulation composition containing a homogeneous mixture of a therapeutic agent of the present invention. When referring to these preformulation enzyme(s) as homogeneous, it is meant that the enzyme(s) is dispersed evenly throughout the composition so that the composition may be readily subdivided into equally effective unit dosage forms, such as tablets, pills and capsules. This solid preformulation is then subdivided into unit dosage forms of the type described herein.

Compressed tablets are solid dosage forms prepared by compacting a formulation containing an active ingredient and excipients selected to aid the processing and improve the properties of the product. The term “compressed tablet” generally refers to a plain, uncoated tablet for oral ingestion, prepared by a single compression or by pre-compaction tapping followed by a final compression.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. A variety of materials can be used for such enteric layers or coatings, including a number of polymeric acids and mixtures of polymeric acids with such materials as shellac, cetyl alcohol and cellulose acetate.

The term “suspension tablets” as used herein refers to compressed tablets which rapidly disintegrate after they are placed in water, and are readily dispersible to form a suspension containing a precise dose of the enzyme(s). Croscarmellose sodium is a known disintegrant for tablet formulations, and is available from FMC Corporation, Philadelphia, Pa. under the trademark Ac-Di-Sol®. It is frequently blended in compressed tableting formulations either alone or in combination with microcrystalline cellulose to achieve rapid disintegration of the tablet.

Microcrystalline cellulose, alone or co-processed with other ingredients, is also a common additive for compressed tablets and is well known for its ability to improve compressibility of difficult to compress tablet materials. It is well known in the art that commercially available products are available and can be used with the present invention. One example is available under the Avicel® trademark. Two different Avicel® products are utilized, Avicel® PH which is microcrystalline cellulose, and Avicel® AC-815, a co processed spray dried residue of microcrystalline cellulose and a calcium-sodium alginate complex in which the calcium to sodium ratio is in the range of about 0.40:1 to about 2.5:1. While AC-815 is comprised of 85% microcrystalline cellulose (MCC) and 15% of a calcium-sodium alginate complex, for purposes of the present invention this ratio may be varied from about 75% MCC to 25% alginate up to about 95% MCC to 5% alginate. Depending on the particular formulation and active ingredient, these two components may be present in approximately equal amounts or in unequal amounts, and either may comprise from about 10% to about 50% by weight of the tablet.

Dry oral formulations can contain such excipients as binders (for example, hydroxypropylmethylcellulose, polyvinyl pyrilodone, other cellulosic materials and starch), diluents (for example, lactose and other sugars, starch, dicalcium phosphate and cellulosic materials), disintegrating agents (for example, starch polymers and cellulosic materials) and lubricating agents (for example, stearates and talc).

Since the tablet may be used to form rapidly disintegrating chewable tablets, lozenges, troches or swallowable tablets; the intermediate formulations, as well as the process for preparing them, provide additional novel aspects of the present invention.

Effervescent tablets and powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid.

When the salts are added to water, the acids and the base react to liberate carbon dioxide gas, thereby causing “effervescence.”

The choice of ingredients for effervescent granules depends both upon the requirements of the manufacturing process and the necessity of making a preparation that dissolves readily in water. The two needed ingredients are at least one acid and at least one base. The base releases carbon dioxide upon reaction with the acid. Examples of such acids include, but are not limited to, tartaric acid and citric acid. In one embodiment, the acid is a combination of both tartaric acid and citric acid. Examples of bases include, but are not limited to, sodium carbonate, potassium bicarbonate and sodium bicarbonate. In one embodiment, the base is sodium bicarbonate, and the effervescent combination has a pH of about 6.0 or higher.

Illustratively, effervescent salts include the following ingredients, which actually produce the effervescence: sodium bicarbonate, citric acid and tartaric acid. When added to water, the acids and base react to liberate carbon dioxide, resulting in effervescence. It should be noted that any acid-base combination that results in the liberation of carbon dioxide could be used in place of the combination of sodium bicarbonate and citric and tartaric acids, as long as the ingredients were suitable for pharmaceutical use and result in a pH of about 6.0 or higher.

It should be noted that it requires three molecules of NaHCO₃ to neutralize one molecule of citric acid and two molecules of NaHCO₃ to neutralize one molecule of tartaric acid. It is desired that the approximate ratio of ingredients is as follows: Citric Acid:Tartaric Acid:Sodium Bicarbonate=1:2:3.44 (by weight). This ratio can be varied and continue to produce an effective release of carbon dioxide. For example, ratios of about 1:0:3 or 0:1:2 are also effective.

The method of preparation of the effervescent granules of the present invention employs three basic processes: wet granulation, dry granulation and fusion. The fusion method is used for the preparation of most commercial effervescent powders. It should be noted that, although these methods are intended for the preparation of granules, the formulations of effervescent salts of the present invention could also be prepared as tablets, according to well-known prior art technology for tablet preparation.

Wet granulation is the oldest method of granule preparation. The individual steps in the wet granulation process of tablet preparation include milling and sieving of the ingredients, dry powder mixing, wet massing, granulation and final grinding.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps.

The liquid forms in which the novel compositions of the present invention may be incorporated for administration orally include aqueous solutions; suitably flavored syrups; aqueous or oil suspensions; and flavored emulsions with edible oils, such as cottonseed oil, sesame oil, coconut oil or peanut oil, as well as elixirs and similar pharmaceutical vehicles.

Other delivery systems can include time-release, delayed-release or sustained-release delivery systems. Such systems can avoid repeated administrations of the enzyme of the present invention, increasing convenience to the subject and the physician. Many types of release delivery systems are available and known to those of ordinary skill in the art. They include polymer-based systems, such as polylactic and polyglycolic acid, polyanhydrides and polycaprolactone; nonpolymer systems that are lipids, including sterols, such as cholesterol, cholesterol esters and fatty acids, or neutral fats, such as mono-, di- and triglycerides; hydrogel release systems; silastic systems; peptide-based systems; wax coatings; compressed tablets using conventional binders and excipients; partially fused implants; and the like. Specific examples include, but are not limited to: (a) erosional systems in which the polysaccharide is contained in a form within a matrix, found in U.S. Pat. No. 4,452,775 (Kent); U.S. Pat. No. 4,667,014 (Nestor et al.); and U.S. Pat. No. 4,748,034 and U.S. Pat. No. 5,239,660 (Leonard) and (b) diffusional systems in which an active component permeates at a controlled rate through a polymer, found in U.S. Pat. No. 3,832,253 (Higuchi et al.) and U.S. Pat. No. 3,854,480 (Zaffaroni).

The present invention is further illustrated by the following formulations, which should not be construed as limiting in any way. The practice of the present invention will employ, unless otherwise indicated, conventional techniques of pharmacology and pharmaceutics, which are within the skill of the art.

The formula per dosage unit of several embodiments are as follows:

### Formula 1—Unit Dose Powder

| | |
|---|---|
| Endo-1,4-beta-xylanase | 10 mg |
| Beta-glucanase | 5 mg |
| Pepsin | 10 mg |
| Excipients and flavoring | 25 mg |
| | 50 mg total per packet |

### Formula 2—Unit Dose Powder

| | |
|---|---|
| Endo-1,4-beta-xylanase | 20 mg |
| Beta-glucanase | 10 mg |
| Hemicellulase | 20 mg |
| Excipients and flavoring | 50 mg |
| | 100 mg total per packet |

This powder is placed into a foil packet, the contents of which may be mixed with about 30 ml to about 120 ml of water prior to oral administration. The powder may also be proportionately bulk compounded and placed in a large (e.g., 1 kg) container. Unit dose scoops of the bulk powder can then be mixed with water to form a solution or suspension.

### Formula 3—Tablet or Capsule

| | |
|---|---|
| Endo-1,4-beta-xylanase (T. longibrachiatum) | 40 mg |
| Beta-glucanase (A. Niger) | 20 mg |
| Protease | 100 mg |
| Hemicellulase | 100 mg |
| Binders and excipients | 100 mg |
| | 360 mg total per tablet |

Illustratively, certain formulations of the present invention may contain about 0.1 mg to about 1000 mg of xylanase, for example, Endo-1,4-beta-xylanase or the equivalent thereof. In another embodiment of the present invention, certain formulations may contain about 1 mg to about 100 mg xylanase, for example, Endo-1,4-beta-xylanase or the equivalent thereof. In another embodiment of the present invention, certain formulations may contain about 0.1, 0.5, 1, 2, 5, 10, 20, 50, 100, 200, 500, or 1000 mg of xylanase, for example, Endo-1,4-beta-xylanase or the equivalent thereof.

In another embodiment of the present invention, certain formulations may contain about 0.1 mg to about 1,000 mg of glucanase, for example, Beta-glucanase or the equivalent thereof. In another embodiment of the present invention, certain formulations may contain about 1 mg to about 100 mg of glucanase, for example, Beta-glucanase or the equivalent thereof. In another embodiment of the present invention, certain formulations may contain about 0.1, 0.5, 1, 2, 5, 10, 20, 50, 100, 200, 500, or 1000 mg of glucanase, for example, Beta-glucanase or the equivalent thereof.

In another embodiment of the present invention, certain formulations may contain about 0.1 mg to about 1,000 mg of a protease or the equivalent thereof. In another embodiment of the present invention, certain formulations may contain about 1 mg to about 100 mg of a protease or the equivalent thereof. In another embodiment of the present invention, certain formulations may contain about 0.1, 0.5, 1, 2, 5, 10, 20, 50, 100, 200, 500, or 1000 mg of a protease or the equivalent thereof.

In another embodiment of the present invention, certain formulations may contain about 0.1 mg to about 1,000 mg of a hemicellulase or the equivalent thereof. In another embodiment of the present invention, certain formulations may contain about 1 mg to about 100 mg of a hemicellulase or the equivalent thereof. In another embodiment of the present invention, certain formulations may contain about 0.1, 0.5, 1, 2, 5, 10, 20, 50, 100, 200, 500, or 1000 mg of a hemicellulase or the equivalent thereof.

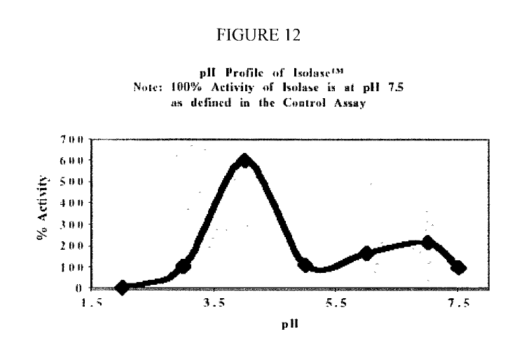

An advantage of the present invention is the high activity of the composition at a pH of about 3.5, which is parallel to stomach acid. An in vitro study was performed to determine the activity of the composition in varying pH environments. The results, as illustrated in FIG. 12, show the peak activity of the composition at about 3.5. This suggests the presence of stomach acid will increase the cleavage and contribute to the in vivo effect.

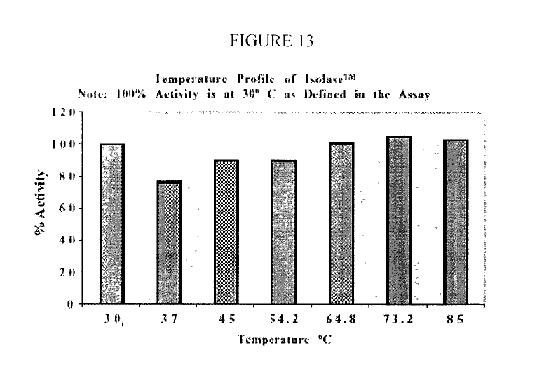

In another embodiment of the present invention, the composition is relatively stable across a wide temperature range. An in vitro study of the composition's activity across a temperature range of about 30-85 degrees Celsius was performed. The results, as illustrated in FIG. 13, show the activity of the composition is at or above 90% for the entire temperature range (excluding about 37 degrees Celsius, which is around 85%).

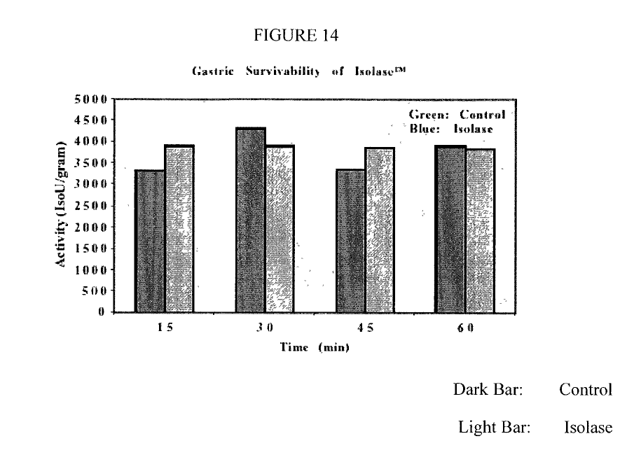

The inventors also measured the activity of the present invention verses the activity of beta-glucosidase. It was determined, as illustrated in FIG. 14, that the present invention's activity is consistent over time and is similar to that of beta-glucosidase.

In another embodiment of the present invention, the invention comes in the form of a kit or package. The present invention can be packaged in the form of a kit or package in which the daily, weekly, or monthly (or other periodic) dosages are arranged for proper sequential or simultaneous administration. The present invention further provides a kit or package containing a plurality of dosage units, adapted for successive daily administration, each dosage unit comprising at least one of the therapeutic compounds of the present invention. This delivery system can be used to facilitate administering any of the various embodiments of the present invention. In one embodiment, the present invention contains a plurality of dosages to be administered daily or weekly. The kits or packages also contain a set of instructions for the subject.

The invention has been described in an illustrative manner, and it is to be understood that the terminology used is intended to be in the nature of description rather than of limitation. All patents and other references cited herein are incorporated herein by reference in their entirety. Obviously, many modifications, equivalents and variations of the present invention are possible in light of the above teachings. Therefore, it is to be understood that, within the scope of the appended claims, the invention may be practiced other than as specifically described.

What is claimed is:

1. A method for converting a glycosylated isoflavone into an aglycone in a digestive tract of a subject in need thereof, comprising: orally administering to the subject an effective amount of a composition comprising at least one enzyme selected from the group consisting of xylanase, glucanase, alpha-galactosidase, lactase and glucosidase; and concomitantly administering to the subject a glycolsylated isoflavone.

2. The method of claim 1, wherein the xylanase is endo-1,4-beta-xylanase.

3. The method of claim 1, wherein the glucanase is beta-glucanase.

4. The method of claim 1, wherein the glucosidase is beta-glucosidase.

5. The method of claim 1, further comprising ingesting the composition concurrently with oral ingestion of a glycoside.

6. The method of claim 1, wherein the glycoside is a flavonoid.

7. The method of claim 6, wherein the flavonoid is an isoflavone.

8. The method of claim 7, wherein the isoflavone is isolated from a source selected from the group consisting of black cohosh, red clover, and soy.

9. The method of claim 1, wherein the aglycone is selected from the group consisting of: genistein, formononetin, benistein, biochanin A, daidzein, and glycetein.

10. The method of claim 1, wherein the composition comprises about two parts endo-1,4-beta-xylanase to about one part beta-glucanase.

11. The method of claim 10, wherein the endo-1,4-beta-xylanase is isolated from T. longibrachiatum.

12. The method of claim 10, wherein the beta-glucanase is isolated from A. niger.

13. The method of claim 1, wherein the composition further comprises a pharmaceutically acceptable carrier.

14. The method of claim 1, wherein the composition further comprises at least one protease.

15. The method of claim 14, wherein the protease is selected from the group consisting of pepsin, papain, trypsin, collagenase, liberase, and a proteolytic enzyme isolated from a plant, an animal or a microbial source.

16. The method of claim 1, wherein the composition further comprises hemicellulase.

17. The method of claim 1, wherein the enzyme combination further comprises a probiotic.

18. A pharmaceutical dosage form, comprising about 5 mg to about 500 mg of a composition comprising a xylanase and a glucanase.

19. The pharmaceutical dosage form of claim 18, wherein the composition comprises about two parts xylanase to about one part glucanase.

20. The dosage form of claim 18, wherein the dosage form is selected from the group consisting of tablet, soft gelatin capsule, hard gelatin capsule, suspension tablet, effervescent tablet, powder, effervescent powder, chewable tablet, solution, suspension, and emulsion.

21. The dosage form as recited in claim 19, wherein the dosage form is a tablet.

22. The dosage form as recited in claim 19, wherein the dosage form is a soft or hard gelatin capsule.

23. The dosage form as recited in claim 19, wherein the dosage form is a suspension tablet.

24. The dosage form as recited in claim 19, wherein the dosage form is an effervescent tablet.

25. The dosage form as recited in claim 19, wherein the dosage form is a powder.

26. The dosage form as recited in claim 19, wherein the dosage form is an effervescent powder.

27. The dosage form as recited in claim 19, wherein the dosage form is a chewable tablet.

28. The dosage form as recited in claim 19, wherein the dosage form is a solution.

29. The dosage form as recited in claim 19, wherein the dosage form is a suspension.

30. The dosage form as recited in claim 19, wherein the dosage form is an emulsion.

31. A method for increasing a serum concentration of an aglycone in a subject in need thereof, comprising: administering to the subject a pharmaceutical dosage form comprising about 5 mg to about 500 mg of a composition comprising a xylanase and a glucanase.

32. The method of claim 31, further comprising ingesting the composition concurrently with ingestion of a glycoside.

33. The method of claim 31, wherein the xylanase is endo-1,4-beta-xylanase.

34. The method of claim 31, wherein the glucanase is beta-glucanase.

35. The method of claim 31, wherein the xylanase is endo-1,4-beta-xylanase and the glucanase is beta-glucanase.

36. The method of claim 31, wherein the composition comprises about two parts endo-1,4-beta-xylanase to about one part beta-glucanase.

37. A method for converting a glycoside into an aglycone in a digestive tract of a subject in need thereof, comprising: orally administering to the subject a composition comprising about 5 mg to about 500 mg endo-1,4-beta-xylanase and beta-glucanase concurrently with ingestion of the glycoside.

38. A method for converting genistin into genistein in a digestive tract of a subject in need thereof, comprising: orally administering to the subject a composition comprising about 5 mg to about 500 mg endo-1,4-beta-xylanase and beta-glucanase concurrently with ingestion of a soy food stuff.

39. A method for converting genistin into genistein in a digestive tract of a subject in need thereof, comprising: orally administering to the subject a composition comprising about 5 mg to about 500 mg of about two parts endo-1,4-beta-xylanase and about one part beta-glucanase concurrently with ingestion of a soy food stuff.

40. A pharmaceutical dosage form, comprising: about 5 mg to about 500 mg of about two parts endo-1,4-beta-xylanase, about one part beta-glucanase, and an optional pharmaceutically acceptable excipient.

41. A method for increasing a serum concentration of an aglycone in a subject in need thereof, comprising: administering to the subject a pharmaceutical dosage form comprising about 5 mg to about 500 mg of about two parts endo-1,4-beta-xylanase and about one part beta-glucanase.

42. A method for increasing a serum concentration of genistein in a subject in need thereof, comprising: administering to a subject a pharmaceutical dosage form comprising about 5 mg to about 500 mg of about two parts endo-1,4-beta-xylanase and about one part beta-glucanase.

43. The pharmaceutical dosage form of claim 40 produced according to the process of
dry granulating the endo-1,4-beta-xylanase, the beta-glucanase, and the optional pharmaceutically acceptable excipient; and
forming the endo-1,4-beta-xylanase, beta-glucanase, and optional pharmaceutically acceptable excipient into a dosage form selected from the group consisting of tablet, soft gelatin capsule, hard gelatin capsule, suspension tablet, effervescent tablet, powder, effervescent powder, and chewable tablet.

44. The pharmaceutical dosage form of claim 40 produced according to the process of
wet granulating the endo-1,4-beta-xylanase, the beta-glucanase, and the optional pharmaceutically acceptable excipient; and
forming the endo-1,4-beta-xylanase, beta-glucanase, and optional pharmaceutically acceptable excipient into a dosage form selected from the group consisting of tablet, soft gelatin capsule, hard gelatin capsule, suspension tablet, effervescent tablet, powder, effervescent powder, and chewable tablet.

45. The pharmaceutical dosage form of claim 40 produced according to the process of
dry mixing the endo-1,4-beta-xylanase, the beta-glucanase, and the optional pharmaceutically acceptable excipient; and
forming the endo-1,4-beta-xylanase, beta-glucanase, and optional pharmaceutically acceptable excipient into a dosage form selected from the group consisting of tablet, soft gelatin capsule, hard gelatin capsule, suspension tablet, effervescent tablet, powder, effervescent powder, and chewable tablet.

46. A process for the manufacture of a pharmaceutical dosage form according to claim 40, comprising:
dry granulating the endo-1,4-beta-xylanase, the beta-glucanase, and the optional pharmaceutically acceptable excipient; and
forming the endo-1,4-beta-xylanase, beta-glucanase, and optional pharmaceutically acceptable excipient into a dosage form selected from the group consisting of tablet, soft gelatin capsule, hard gelatin capsule, suspension tablet, effervescent tablet, powder, effervescent powder, and chewable tablet.

47. A process for the manufacture of a pharmaceutical dosage form according to claim 40, comprising:
wet granulating the endo-1,4-beta-xylanase, the beta-glucanase, and the optional pharmaceutically acceptable excipient; and
forming the endo-1,4-beta-xylanase, beta-glucanase, and optional pharmaceutically acceptable excipient into a dosage form selected from the group consisting of tablet, soft gelatin capsule, hard gelatin capsule, suspension tablet, effervescent tablet, powder, effervescent powder, and chewable tablet.

48. A process for the manufacture of a pharmaceutical dosage form according to claim 40, comprising:
dry mixing the endo-1,4-beta-xylanase, the beta-glucanase, and the optional pharmaceutically acceptable excipient; and
forming the endo-1,4-beta-xylanase, beta-glucanase, and optional pharmaceutically acceptable excipient into a dosage form selected from the group consisting of tablet, soft gelatin capsule, hard gelatin capsule, suspension tablet, effervescent tablet, powder, effervescent powder, and chewable tablet.
